(19) United States
(12) Patent Application Publication
(10) Pub. No.: US 2013/0197400 A1
(43) Pub. Date: Aug. 1, 2013

(54) System and method for determining the optimum size of an ultrasonic wave generation device

(71) Applicant: Eye Tech Care, Rillieux-La-Pape (FR)

(72) Inventors: Fabrizio ROMANO, Beynost (FR); Florent APTEL, Laxou (FR); Laurent FARCY, Liergues (FR)

(73) Assignee: EYE TECH CARE, Rillieux-La-Pape (FR)

(21) Appl. No.: 13/707,671

(22) Filed: Dec. 7, 2012

(30) Foreign Application Priority Data
Dec. 8, 2011 (FR) 1161309

(51) Int. Cl. A61N 7/00

(52) U.S. Cl. CPC A61N 7/00
USPC 601/2

(57) ABSTRACT

The present invention relates to a method for determining a probe model for treating an eye pathology, from among a plurality of different probe models, each probe including a cone-shaped ring including a proximal end (11) intended to be in contact with one eye (4) of a patient and a distal end (12) intended to receive means for generating ultrasonic waves (2), the method comprising a step for receiving at least one measurement of a biometric parameter of the eye, and a step for determining the optimum probe model according to each biometric parameter.

### CROSS REFERENCE TO RELATED APPLICATIONS

This application claims priority to French Application 1161309 filed Dec. 8, 2011.

### BACKGROUND OF THE INVENTION

1. Field of the Invention

The present invention relates to the general technical field of non-invasive treatment of an eye pathology.

More particularly, it relates to a system and method allowing determination of the optimum size of a probe of a device for generating high intensity focused ultrasonic waves for treating such an eye pathology like glaucoma.

2. Description of Related Art

Glaucoma is an optical neuropathy, i.e. a disorder of the optical nerve, due to high intraocular pressure (10P).

The eye is a hollow structure consisting of two segments: the anterior segment between the cornea and the crystalline lens, and the posterior segment between the crystalline lens and the retina. The anterior segment contains a transparent liquid called “the aqueous humor”.

The aqueous humor is formed in the posterior chamber of the anterior segment of the eye by the ciliary body. The liquid, which is generated at a relatively constant rate, then passes around the crystalline lens, through the pupillary aperture of the iris and into the anterior chamber of the eye. The aqueous humor is then mainly discharged through the trabeculum and Schlemm's canal.

When the aqueous humor is no longer sufficiently rapidly discharged, the latter accumulates, which induces an increase in the IOP. The increase in the IOP compresses the axons in the optical nerve and may also compromise vascularization of the optical nerve. A high IOP for a long period may induce a total loss of vision.

The only therapeutic approach presently available for treating glaucoma consists of reducing the intraocular pressure:
either by improving drainage of the aqueous humor,
or by reducing the production of aqueous humor.

From document WO 2009/103721, a device is known for reducing the production of aqueous humor based on the cyclo-coagulation principle by high intensity focused ultrasonic waves which consists of destroying part of the ciliary bodies in order to reduce the production of aqueous humor.

With the device described in WO 2009/103721, it is possible to treat glaucoma in one step.

With reference to FIGS. 1 and 2, this device 1 comprises a probe consisting of a ring and of means for generating ultrasonic waves. The ring has a proximal portion intended to be in contact with one eye and a distal portion intended to receive the means for generating focused high intensity ultrasonic waves. The means for generating ultrasonic waves comprise six transducers with a concave profile with the shape of a cylinder segment, positioned on a cylindrical crown.

The dimensions of the ring have to be adapted according to the dimensions of the eye of the patient to be treated in order to allow accurate positioning of the means for generating ultrasonic waves so as to selectively and specifically destroy a portion of the ciliary body and to spare the adjacent structures.

From the document entitled <<miniaturized High-Intensity Focused Ultrasound Device in Patients with Glaucoma: A Clinical Pilot Study>>, a method is known giving the possibility of selecting a probe model from a plurality of probe models. In order to select the most suitable probe model for treating an eye, this document proposes the use of an image by ultrasound biomicroscopy.

A drawback of this method is that it requires the use of an imaging device by ultrasound microscopy, a device not very widespread and expensive.

### SUMMARY

An object of the present invention is to propose a system and a method allowing determination of the optimum dimensions of the probe of the device described in WO 2009/103721 according to the dimensions of the eye of the patient, this system and this method not requiring the use of an expensive imaging device.

For this purpose, the invention proposes a method for determining an optimum probe model for treating an eye pathology, from a plurality of different probe models, each probe including a ring with a conical shape including a proximal end suitable to be in contact with one eye of a patient and with a distal end suitable to receive means for generating ultrasonic waves, the method comprising the following steps:
receiving at least one measurement of at least one biometric parameter of the eye,
determining the optimum probe model according to said and at least one biometric parameter.

### BRIEF DESCRIPTION OF THE DRAWINGS

FIGS. 1-6 depict embodiments of the present invention.

### DETAILED DESCRIPTION OF A PREFERRED EMBODIMENT

The models of probes from the plurality of probe models may be different in that they include rings of different dimensions.

Within the scope of the present invention by <<biometric parameter of the eye >>, is meant a dimension of a portion of the eye such as:
the width (i.e. the diameter) of the iris,
the axial length of the eye,
the radius of curvature of the sclera,
the thickness of the transparent cornea,
the anterior or posterior radius of the crystalline lens,
the diameter of the crystalline lens, the thickness of the crystalline lens.

In all the cases, the received measurement(s) is (are) without any ultrasonic biomicroscopy image of the eye to be treated. In other words, these measurement(s) does (do) not contain any ultrasonic biomicroscopy image of the eye to be treated.

The determination of an optimum probe model may comprise the selection from a plurality of probe models, of the most suitable probe model for the treatment to be applied. This selection depends on the measurement(s) of the biometric parameter(s).

For example, when the means for generating ultrasonic waves of the probe comprise at least one transducer laid out so as to generate focused high intensity ultrasonic waves, then the optimum probe model is the one for which the distance between the area to be treated and the focusing area of the transducer is minimum when said optimum probe model is set into place on the eye to be treated.

Preferred but non-limiting aspects of the device according to the invention are the following:
the reception step comprises the reception of a measurement of the horizontal width of the iris passing through the center of the pupil, a so-called white-to-white distance of the eye;
the use of a measurement of the horizontal width of the iris gives the possibility of determining the optimum probe to be used for treating the eye from a simple eye examination only requiring the use of rudimentary instruments such as a ruler,
the step for determining the optimum probe model comprises the following sub-steps:
   If the white-to-white distance is less than a first threshold, for example less than 11 millimeters, then a first probe model is selected,
   If the white-to-white distance is comprised between the first threshold and a second threshold, for example comprised between 11 and 12 millimeters, a second probe model is selected,
   If the white-to-white distance is greater than the second threshold, for example greater than 12 millimeters, a first probe model is selected, the first, second and third models of probes comprising rings with different dimensions;
the reception step comprises the reception of a measurement of the axial length of the eye;
the use of a measurement of the axial length of the eye allows determination of the optimum probe to be used for treating the eye, from a simple eye examination requiring common eye instruments available to the majority of the practitioners,
wherein the biometric parameter of the eye is selected from:
   the horizontal width of the iris passing through the center of the pupil, and/or
   the axial length of the eye
the optimum probe model being determined according to the horizontal width of the iris passing through the center of the pupil, and/or to the axial length of the eye.
the combined use of a measurement of the axial length of the eye and of a measurement of the horizontal width of the iris gives the possibility of refining the accuracy in the determination of the optimum probe model.
the step for determining the optimum probe model comprises the following substeps:
   If the axial length of the eye is less than a first value, for example less than 23 millimeters, then a first probe model is selected,
   If the axial length is comprised between the first value and a second value, for example comprised between 23 and 25 millimeters, a second probe model is then selected,
   If the axial length is greater than the second value, for example greater than 25 millimeters, a third probe model is selected,
the first, second and third models of probes comprising rings of different dimensions;
the reception step comprises the reception of a measurement of the white-to-white distance of the eye and the reception of a measurement of the axial length of the eye, the step for determining the optimum probe model comprises the following substeps:
   If the axial length distance of the eye is less than a fourth value, for example less than 22 millimeters, a first probe model is then selected,
   If the axial length of the eye is comprised between the first and the fourth value, for example comprised between 22 millimeters and 23 millimeters, and if the white-to-white distance is less than the first threshold, for example greater than 11 millimeters, a first probe model is then selected,
   If the axial length of the eye is comprised between the first and the fourth value, for example comprised between 22 millimeters and 23 millimeters and if the white-to-white distance is greater than the first threshold, for example greater than 11 millimeters, a second probe model is then selected,
   If the axial length is comprised between the first value and a fifth value, for example comprised between 23 and 24.5 millimeters, a second probe model is then selected,
   If the axial length of the eye is comprised between the fifth value and a sixth value, for example comprised between 24.5 millimeters and 25.5 millimeters and if the white-to-white distance is less than the second threshold, for example less than 12 millimeters, a second probe model is then selected,
   If the axial length of the eye is comprised between the fifth value and a sixth value, for example comprised between 24.5 millimeters and 25.5 millimeters and if the white-to-white distance is greater than the second threshold, for example greater than 12 millimeters, a third probe model is then selected,
   If the axial length is greater than the sixth value, for example greater than 25.5 millimeters, a third probe model is selected,
the first, second and third models of probes comprising rings of different dimensions.
the method further comprises the following steps:
   receiving a representative image of the anterior segment of the eye such as an ultrasonic biomicroscopy image,
   determining the region containing the ciliary bodies in the representative image of the anterior segment of the eye,
   superposing the representative image of the anterior segment of the eye to a plurality of template images, optionally after a step for setting the scale of the images, so as to obtain a plurality of superposed images, each template image representing at least the focusing area of the ultrasonic waves for a respective probe model,
   estimating the distance between the focusing area and the region containing the ciliary bodies,
   selecting the probe model for which the distance between the focusing area and the region containing the ciliary bodies is minimum.
The fact of using an ultrasonic biometry image in addition to the biometric parameter(s), gives the possibility of improving the accuracy in the selection of the optimum probe model.

One skilled in the art will appreciate that the dimensions indicated above with reference to the different biometric parameters (11, 12, 22, 23, 24, 24.5, 25.5 etc.) are to be taken into account with a margin of the order of more or less 0.2 (or even 0.4) and which corresponds to the measurement uncertainty.

The invention also relates to a computer program product comprising instructions of a program code recorded on a medium which may be used in a computer, characterized in that it comprises instructions for applying the method described above.

The invention also relates to a system for determining a probe model for treating an eye pathology from a plurality of different probe models, each probe including a ring including a proximal end intended to be in contact with one eye of a patient and a distal end intended to receive means for generating ultrasonic waves, the system comprising a computer programmed so as to:
receive at least one measurement of a biometric parameter of the eye,
determine an optimum probe model depending on each biometric parameter.

Other advantages and features will become better apparent from the description which follows of several alternative embodiments, given as non-limiting examples, from the appended drawings wherein:

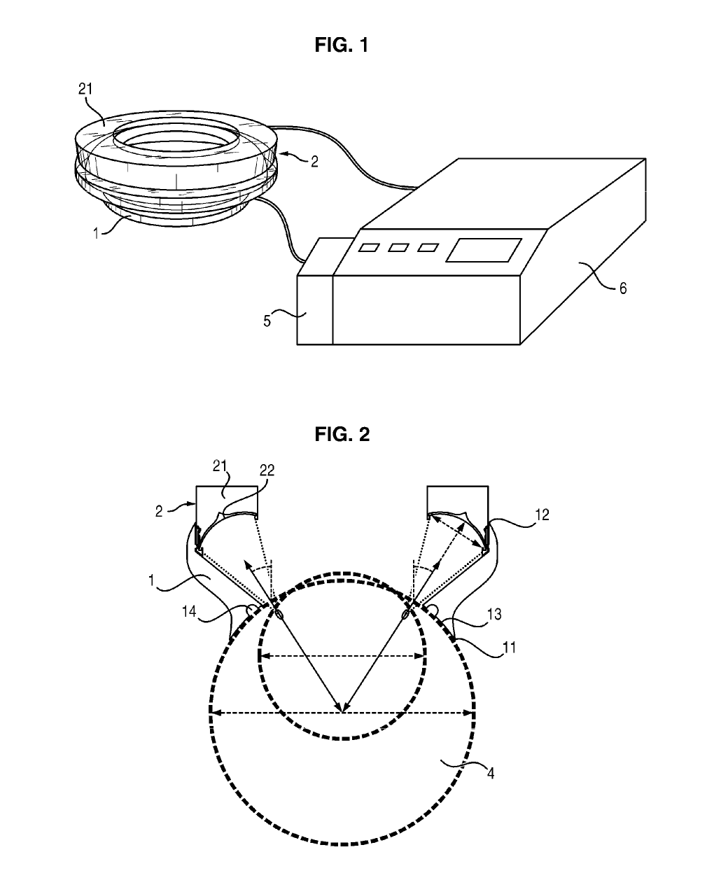

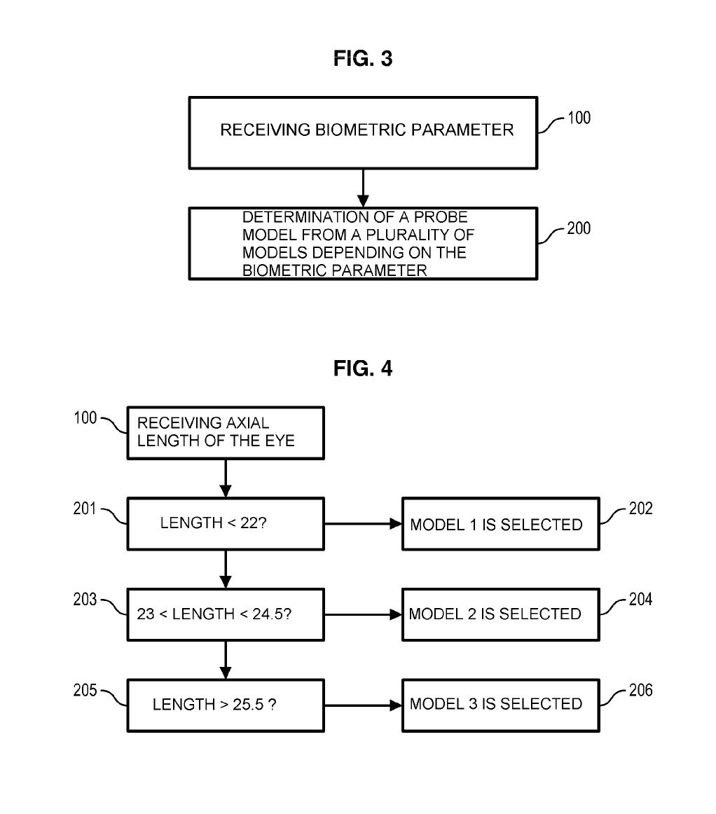

FIGS. 1 and 2 illustrate an embodiment of the device described in WO 2009/103721;

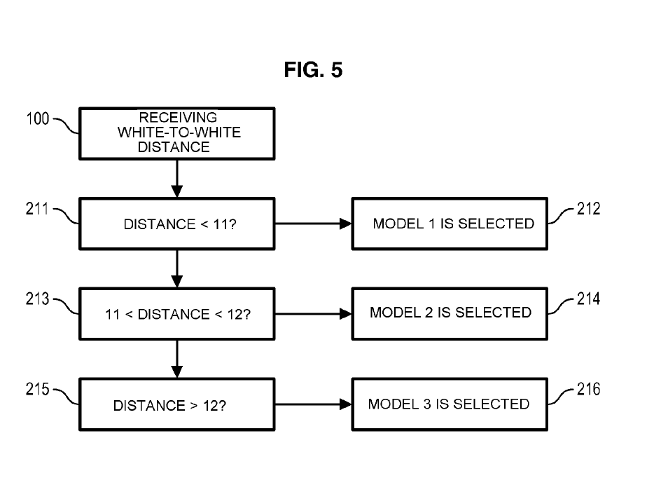

FIGS. 3 to 7 illustrate various alternatives of the method according to the invention,

FIGS. 8 and 9 illustrate template and final images.

With reference to FIGS. 1 and 2, an embodiment of the device for treating an eye pathology, described in WO 2009/103721, was illustrated.

The device comprises a probe consisting of:
a ring 1,
means for generating ultrasonic waves 2 for generating focused ultrasonic waves.

This device allows the treatment of glaucoma in one go.

### Ring

The ring 1 consists in a cone frustum open at both ends. The small base 11 of the cone frustum is the proximal end of the ring 1, and the large base 12 is the distal end of the ring 1.

The ring 1 allows adequate and constant positioning of the means for generating ultrasonic waves 2, both for centering and for the distance relatively to the sclera of said means for generating ultrasonic waves 2.

The proximal end 11 is intended to come into contact with one eye 4 of a patient. The distal end 12 of the ring 1 is intended to receive the means for generating ultrasonic waves 2.

With reference to FIG. 1, the proximal end 11 of the cone frustum comprises an external ring-shaped flange 13 able to be applied on the external surface of the eye 4, at about 2 mm from the limb, the limb being the junction between the cornea and the sclera.

The proximal edge 11 of the cone frustrum also includes an annular groove 14 connected to a suction device 5 through at least one aperture passing through the cone frustrum 1 and opening into the annular groove 14. Advantageously, the suction device 5 may be controlled by a control unit 6.

It is obvious that the suction device 5 may be independent without departing from the scope of the invention.

The operating principle of the suction device 5 is the following. The cone frustrum 1 is applied on the eye 4 of the patient and the suction device 5 is activated. The latter induces the production of a depression in the annular groove 14 which causes deformation of the conjunctiva of the eye 4, this deformation forming an O-ring gasket in the annular groove 14.

The cone frustrum 1 is then closely bound to the eye 4 so that the cone frustrum 1 will follow micromovements of the eye 4 during the duration of the treatment. This gives the possibility of ensuring that the position of the apparatus is maintained centered on the visual axis.

### Means for Generating Ultrasonic Waves

The means for generating ultrasonic waves 2 allow generation of ultrasound energy. The means for generating ultrasonic waves include a cylindrical crown 21 on which transducer(s) 22 is (are) laid out, suitable for generating ultrasonic waves.

In certain embodiments, the profile of the transducer(s) is suitable for allowing orientation and focusing of the ultrasonic waves in a given point. In other embodiments, the transducer(s) is (are) associated with reflector(s) allowing reflection, orientation and focusing of the generated ultrasonic waves in a given point.

The means for generating ultrasonic waves 2 are connected to the control unit 6. The latter includes a salvo generator and means for specifying the parameters of the salvo such as the frequency, the power and the duration of each burst, etc.

The salvo generator comprises at least one sinusoidal signal generator at a determined frequency comprised between 5 and 25 MHz, and preferably between 19 and 22 MHz, an amplifier and an electric counter.

### General Points Relating to the Use of the Device Illustrated in FIGS. 1 and 2

The goal of the device described in WO 2009/103721 is to selectively destroy a portion of the ciliary bodies and to spare the adjacent structures. To do this, the device described in WO 2009/103721 is based on the generation of high intensity ultrasonic waves focused on the ciliary bodies. This focusing of the ultrasonic waves in a given point is obtained by adapting the dimensions and the orientation of the rings and of the means for generating ultrasonic waves.

As the dimensions of the human eye may vary from one individual to the other, the inventors of the device described in WO 2009/103721 propose different dimensions of probes (ring+means for generating ultrasonic waves) in order to allow optimum positioning of the focusing point of the ultrasonic waves at the ciliary bodies of the eye to be treated.

Thus, the inventors of the device in WO 2009/103721 propose four different models of probes each consisting of a ring and of a crown in order to meet the inter-individual anatomic variability:
a first model wherein the ring has a diameter of 10 millimeters,
a second model wherein the ring has a diameter of 11 millimeters,
a third model wherein the ring has a diameter of 12 millimeters,
a fourth model wherein the ring has a diameter of 13 millimeters.

For these four probe models, the dimensions and the orientation of the means of generating ultrasonic waves are adapted so as to allow optimum positioning of the focusing area at the ciliary body to be treated.

An object of the invention is to allow determination of the optimum probe model to be used for treating a patient according to the dimensions of his/her eye.

In the embodiment of the invention shown in the following, it will be assumed that the determination of the optimum probe model has to be accomplished from the three models of probes described earlier. However, it is quite obvious that the invention may be applied on a smaller or on a larger number of different probe models.

With reference to FIG. 3, an embodiment of the method according to the invention was illustrated.

This method comprises a first step 100 for receiving measurement(s) of biometric parameter(s).

A second step 200 of the method consists of selecting from a plurality of different probe models, the probe for which the dimensions are the most adapted to the patient. This selection is determined according to the biometric parameter(s) received in the previous step.

Thus, the method illustrated in FIG. 3 proposes determination of the optimum probe model depending on evaluated biometric parameter(s) of the eye, during a routine clinical examination (white-to-white diameter, etc), or with a simple para-clinical examination (axial length, objective refraction, etc.).

This allows a reduction in the costs related to the determination of the optimum probe model by doing away with the use of an imaging device by ultrasonic biomicroscopy, a not very widespread apparatus because of the high costs of the latter.

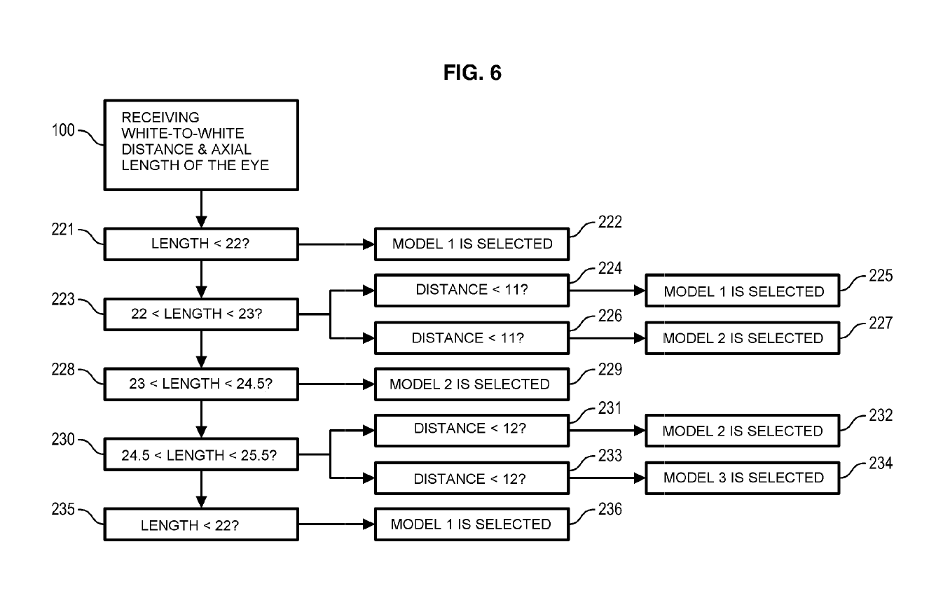

With reference to FIG. 4, an example of a step for determining an optimum probe was illustrated. In this example, the biometric parameter taken into account for determining the optimum probe model from the plurality of probe models is the axial length of the eye.

More specifically:
If the axial length of the eye is less than 22 millimeters (step 201) a first probe model is then selected (step 202),
If the axial length is comprised between 23 and 24.5 millimeters (step 203), a second probe model is then selected (step 204),
If the axial length is greater than 25.5 millimeters (step 205), a third probe model is selected (step 206).

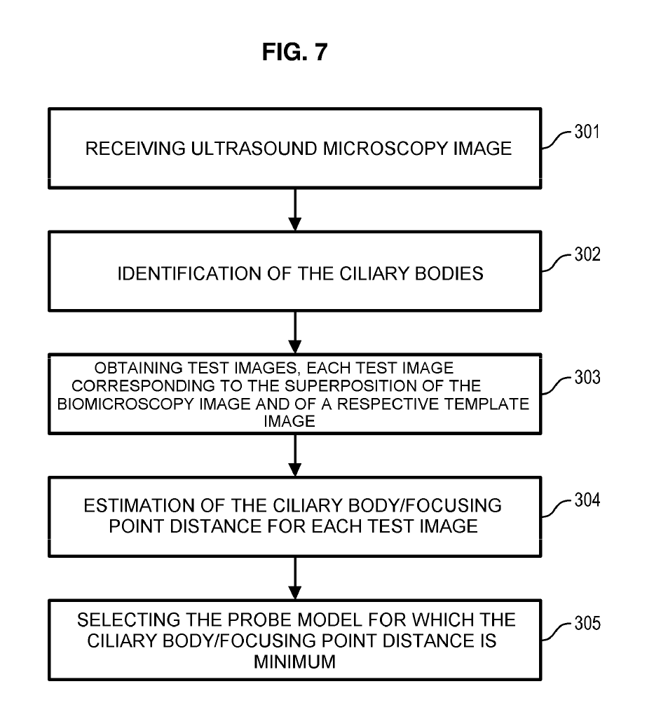

With reference to FIG. 5, another example of a step for determining an optimum probe was illustrated. In this example, the biometric parameter taken into account for determining the optimum probe model from the plurality of probe models is the horizontal width of the iris, a so-called white-to-white distance of the eye.

More specifically:
If the white-to-white distance is less than 11 millimeters (step 211), a first probe model is then selected (step 212),
If the white-to-white distance is comprised between 11 and 12 millimeters (step 213), a second probe model is then selected (step 214),
If the white-to-white distance is greater than 12 millimeters (step 215), a third probe model is then selected (step 216).

With this method, it is possible to determine an optimum probe model from a measurement easy to apply, i.e. of the white-to-white distance.

In order to improve the validity rate of the optimum probe selection from the plurality of probe models, the two biometric parameters mentioned earlier—i.e. the axial length of the eye and the white-to-white distance—may be combined.

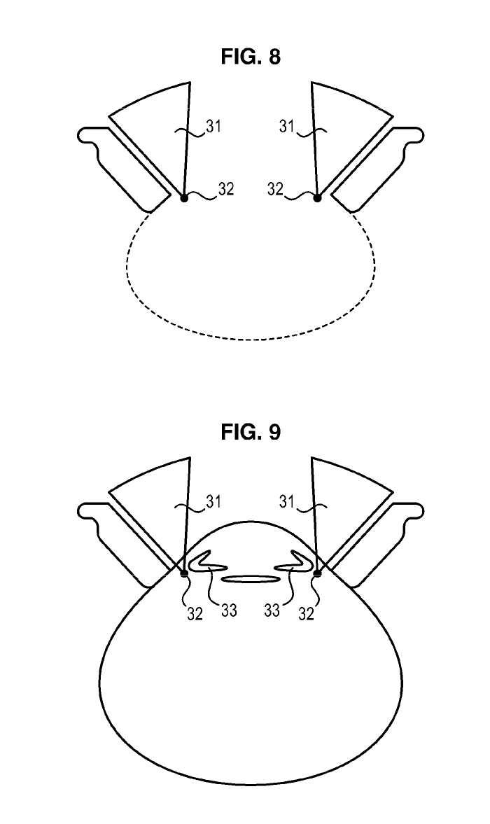

More specifically and with reference to FIG. 6, the step for determining an optimum probe may comprise the following substeps:
If the axial length distance of the eye is less than 22 millimeters (step 221), a first probe model is then selected (step 222),
If the axial length of the eye is comprised between 22 millimeters and 23 millimeters (step 223) and if the white-to-white distance is less than 11 millimeters (step 224), a first probe model is then selected (step 225),
If the axial length of the eye is comprised between 22 millimeters and 23 millimeters (step 223) and if the white-to-white distance is greater than 11 millimeters (step 226), a second probe model is then selected (step 227),
If the axial length is comprised between 23 and 24.5 millimeters (step 228) a second probe model is then selected (step 229),
If the axial length of the eye is comprised between 24.5 millimeters and 25.5 millimeters (step 230) and if the white-to-white distance is less than 12 millimeters (step 231), a second probe model is then selected (step 232),
If the axial length of the eye is comprised between 24.5 millimeters and 25.5 millimeters (step 230) and if the white-to-white distance is greater than 12 millimeters (step 233), a third probe model is then selected (step 234),
If the axial length is greater than 25.5 millimeters (step 235), a third probe model is selected (step 236).

In the case when an ultrasonic biomicroscopy image of the eye of the patient to be treated is available, it is possible to apply the embodiment of the method illustrated in FIG. 7.

With reference to FIG. 7, the method comprises the following steps:
receiving an image by ultrasonic biomicroscopy (step 301),
determining the region containing the ciliary bodies in the ultrasonic biomicroscopy image (step 302),
superposing the duplicated image to a plurality of template images in order to obtain a plurality of final images, each template image illustrating at least the focusing area of the ultrasonic waves for a respective probe model (step 303),
estimating the distance between the focusing area and the region containing the ciliary bodies in each final image (step 304),
selecting the probe model for which the distance between the focusing area and the region containing the ciliary bodies is minimum (step 305).

An exemplary template image was illustrated in FIG. 9 and an exemplary final image in FIG. 10. Each template image may comprise a partial or complete illustration of the probe—i.e. of the ring and of the crown—as well as a partial or complete illustration of the beam of ultrasonic waves 30 generated by the means for generating ultrasonic waves 2. This allows the user to visually check for each final image corresponding to the superposition of the ultrasonic biomicroscopy image to a respective template image, whether the position of the ciliary body 33 of the eye to be treated coincides with the focusing point 32.

The determination of the region containing the ciliary bodies may be applied by any processing method known to one skilled in the art, such as a morpho-mathematical method based on thresholding, etc.

The method may comprise a step for redimensioning the image by ultrasonic biomicroscopy and of the template image so that both of these images are at the same scale. This step may be applied by any technique known to one skilled in the art.

Also, the steps for superposition of the images and estimation of the distance between the ciliary bodies and the focusing point of the ultrasonic waves may be applied by any technique known to one skilled in the art.

The method described earlier may be applied in a processing system comprising a programmed computer for executing the different steps of the method.

The computer is for example computer(s), processor(s), microcontroller(s), microcomputer(s), programmable automaton(a), specific application integrated circuit(s), other programmable circuits, or other devices which include a computer such as workstation.

The computer is coupled with memory(ies) which may be integrated to or separated from the computer. The memory may be a ROM/RAM memory of the computer, a CD-ROM, a USB stick, a memory of a central server. This memory may allow the storage:
of images by ultrasonic biomicroscopy, template images, and final images or further
the processing method applied by the computer.

One skilled in the art will have understood that many modifications may be brought to the device described above without materially departing from the novel teachings shown here.
For example, in the preceding description, the device and the method were shown as allowing selection of an optimum model from among three different models according to the dimension of the eye to be treated. It is quite obvious that the system and the method may allow determination of an optimum model from among less than three different models (for example two), or more than three different models (four, five, . . . or <<n>>models having ring dimensions which may vary between 1 and 100 millimeters for the treating of children or adults). For example, the method described earlier may be used for determining an optimum model from four models: a first model wherein the ring has a diameter of 10 millimeters,
a second model wherein the ring has a diameter of 11 millimeters,
a third model wherein the ring has a diameter of 12 millimeters, or
a fourth model wherein the ring has a diameter of 13 millimeters.

It is therefore quite obvious that the examples which have just been given are only particular illustrations which are by no means limiting.

For example, instead of receiving a measurement of a biometric parameter of the eye (length or width of the iris, etc.) for determining an optimum probe model, the method may comprise the reception of a value of the biometric parameter corresponding to an average computed from a plurality of measurements of said biometric parameter.

Alternatively, the method may comprise:
receiving a plurality of measurements of a biometric parameter,
computation of an average value from this plurality of measurements, and
determining a probe model the most adapted to the eye to be treated from this calculated average value.

1. A method for determining an optimum probe model for treating an eye pathology from among a plurality of different probe models, each probe including a cone-shaped ring including a proximal end suitable to be in contact with one eye of a patient and a distal end suitable to receive means for generating ultrasonic waves,
wherein the method comprises:
receiving at least one measurement of at least one biometric parameter of the eye,
determining an optimum probe model according to said at least one biometric parameter.

2. The method according to claim 1, wherein the biometric parameter of the eye is selected from:
the horizontal width of the iris passing through the center of the pupil, and/or
the axial length of the eye
the optimum probe model being determined according to the horizontal width of the iris passing through the center of the pupil, and/or the axial length of the eye.

3. The method according to claim 1, wherein the receiving comprises reception of at least one measurement of the horizontal width of the iris passing through the center of the pupil, optionally comprising a white-to-white distance of the eye.

4. The method according to claim 3, wherein said determining the optimum probe model comprises the following:
If the white-to-white distance is less than 11 millimeters, then selecting a first probe model,
If the white-to-white distance is comprised from 11 to 12 millimeters, then selecting a second probe model,
If the white-to-white distance is greater than 12 millimeters, then selecting a third probe model,
the first, second and third models of probes comprising rings with different dimensions.

5. The method according to claim 1, wherein the receiving comprises reception of at least one measurement of the axial length of the eye.

6. The method according to claim 5, wherein said determining the optimum probe model comprises the following:
If the axial length of the eye is less than 23 millimeters, then selecting a first probe model,
If the axial length is comprised from 23 to 25 millimeters, then selecting a second probe model,
If the axial length is greater than 25 millimeters, then selecting a third probe model,
the first, second and third models of probes comprising rings with different dimensions.

7. The method according to claim 1, wherein the receiving comprises reception of at least one measurement of the white-to-white distance of the eye and reception of at least one measurement of the axial length of the eye, said determining the optimum probe model comprising the following:
If the axial length distance of the eye is less than 22 millimeters, then selecting a first probe model,
If the axial length of the eye is comprised from 22 millimeters to 23 millimeters and the if the white-to-white distance is less than 11 millimeters, then selecting a first probe model,
If the axial length of the eye is comprised from 22 millimeters to 23 millimeters and if the white-to-white distance is greater than 11 millimeters, then selecting a second probe model is selected,
If the axial length is comprised from 23 to 24.5 millimeters, then selecting a second probe model,
If the axial length of the eye is comprised from 24.5 millimeters to 25.5 millimeters and if the white-to-white distance is less than 12 millimeters, then selecting a second probe model,
If the axial length of the eye is comprised from 24.5 millimeters to 25.5 millimeters and if the white-to-white distance is greater than 12 millimeters, then selecting a third probe model,
If the axial length is greater than 25.5 millimeters, then selecting a third probe model,
the first, second and third models of probes comprising rings with different dimensions.

8. The method according to claim 1, which further comprises the following:
receiving an image representative of an anterior segment of the eye,
determining a region comprising ciliary bodies in the image representative of the anterior segment of the eye,
superposing the representative image of the anterior segment of the eye to a plurality of template images so as to obtain a plurality of superposed images, each template image illustrating at least the focusing area of the ultrasonic waves for a respective probe model,
estimating the distance between focusing area and the region containing the ciliary bodies for each superposed image,
selecting the probe model for which the distance between the focusing area and the region containing the ciliary bodies is minimum.

9. A computer program product comprising program code instructions recorded on a medium which may be used in a computer, wherein the computer program product comprises instructions for applying the method according to claim 1.

10. A system for determining a probe model for treating an eye pathology from among a plurality of different probe models, each probe including a ring including a proximal end suitable to be in contact with one eye of a patient and a distal end suitable to receive means for generating ultrasonic waves,
wherein the system comprises a programmed computer for:
receiving at least one measurement of at least one biometric parameter of the eye, determining an optimum probe model depending on said and on at least one biometric parameter.
